(19) United States
(12) Patent Application Publication
(10) Pub. No.: US 2010/0087749 A1
(43) Pub. Date: Apr. 8, 2010

(54) Collection Device for Sampling Exhaled Airstreams

(76) Inventor: Euan Roger Tovey, New South Wales (AU)

Correspondence Address: COOK ALEX LTD, SUITE 2850, 200 WEST ADAMS STREET, CHICAGO, IL, 60606, US

(21) Appl. No.: 12/524,781

(22) PCT Filed: Jan. 31, 2008
(86) PCT No.: PCT/AU2008/000100

§ 371 Date: Jul. 28, 2009

(30) Foreign Application Priority Data
Jan. 31, 2007 (AU) 2007900458
Jul. 25, 2007 (AU) 2007903984

(51) Int. Cl. A61B 5/097

(52) U.S. Cl. 600/543

(57) ABSTRACT

A collection device (10), such as a mask, for collecting constituents of interest present in an airstream exhaled by a subject The collection device comprises a support member (12) mountable external to the nose and/or mouth of the subject, and at least one collection region (13) mounted to the support member (12) for collecting at least some of said constituents. The constituents are collected in a manner that allows subsequent analysis of the constituents. When the support member (12) is mounted over the nose and/or mouth of the subject, at least some of the exhaled airstream passes through the at least one collection region (13).

### CROSS-REFERENCE TO RELATED APPLICATIONS

The present application claims priority from Australian Provisional Patent Application No 2007900458 filed on 31 Jan. 2007 and Australian Provisional Patent Application No 2007903984 filed on 25 Jul. 2007, the contents of which are incorporated herein by reference.

### FIELD OF THE INVENTION

The present invention relates to a device and method for collecting desired constituents, such as molecules, particles or microorganisms, present in an exhaled airstream.

### BACKGROUND OF THE INVENTION

Many diseases, such as the common cold, can be transmitted by droplets generated by a person coughing and sneezing. One way this transmission occurs is when a person with a cold sneezes, which generates tiny droplets of mucous carrying the infective virus, and these airborne droplets are dispersed into the air. These droplets are subsequently inhaled by another person and deposited in the airways of that person where the virus replicates, leading to symptoms of a cold in the host person.

There are many infectious organisms, both bacterial and viral, that can be spread person-to person, or between species, by this or similar aerosol routes. Examples include rhinovirus, para-influenza virus, and influenza virus and Mycobacterium tuberculosis (TB). In addition to these bacteria or viruses, there are many other micro-organisms or molecules of biological or of diagnostic significance that may also be present in aerosolised droplets or in a gaseous form occurring in the exhaled air.

Persons can also spread some of these diseases even when they do not appear to have symptoms. It is thought that up to 30% of influenza is spread by people in the early stages of infection who do not appear to have symptoms but who are shedding virus into the air.

Determination of the shedding of airborne virus or other micro-organisms by individuals is not usually performed. As well, the quantities of the micro-organisms involved may be small, variable and difficult to sample and detect with some conventional methods. Instead, with cases where a viral infection of the upper airways is suspected, samples of mucous are usually directly collected from the airways. There are several methods of sampling such mucous. The techniques include collecting a sample of mucous obtained from blowing the nose, wiping part of the surface of the nasal cavity or throat with a swab, washing out the nose and throat (lavage) and finally collecting the mucous from the nasal cavity using a device to suction a sample of mucous. The last practice is called ‘nasopharyngeal aspiration’ or NPA, and is commonly used by medical professionals in an appropriate medical setting where all the facilities required are available. The technique is invasive and is commonly only performed on young children up until the age of 2 to 4 years as it is very uncomfortable for adults. In both cases it is generally only performed where there is a sound clinical reason.

Samples can also be collected from the lower airways and methods for this include inducing sputum and collecting it, or performing a technique called bronchoscopy whereby a tube is passed into the lower airways to provide access and enable sampling by suction, brushing, or lavage (in this case the procedure is termed bronchial alveolar lavage or BAL). The techniques of NPA and BAL provide information about virus at the sampling site in the airways, but do not provide information about whether virus is being expelled by the subject.

Exhaled Breath Condensate (EBC) is another diagnostic sampling method which involves the collection by condensation of airway fluid, condensed vapour and soluble volatiles from the airways. This is done by passing exhaled air through a freezing chamber so these components are condensed and can then be collected. This method is generally performed in specialist facilities, often within medical research or specialist hospital departments.

The present invention is directed to devices and methods for improving the collection of desired constituents in an exhaled airstream of a subject, in a non-invasive manner and without necessarily requiring use of specialist facilities.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

According to one aspect, the present invention is a collection device for collecting constituents present in an airstream exhaled by a subject, the collection device comprising:

a support member mountable over the nose and/or mouth of the subject; and

at least one collection region mounted to the support member for collecting at least some of said constituents and holding said constituents in the collection region in a manner that allows subsequent analysis of said constituents;

wherein when the support member is mounted over the nose and/or mouth of the subject, at least some of said exhaled airstream passes through said at least one collection region.

In one embodiment, the collection device can comprise a mask, mask-like device, or an oro-nasal device. In this regard, it is to be understood that if a mask is used, it is positioned over the nose and/or mouth of the subject and does not require use of orifice penetrating members, such as nasal tubes. In another embodiment, the device may include a member, such as a tube or tubes, that are fitted into an oral and/or nasal cavity. Where used, the tube can feed an exhaled airstream to the at least one collection region. In this embodiment, the at least one collection region can be positioned within the tube, at a proximal or distal end of the tube or be positioned external the tube on the support member.

In one embodiment, the constituents comprise molecules, particles and/or organisms. In one embodiment, the collection region can be suitable for collecting respiratory droplets, aerosols and/or other particles exhaled by the subject.

The collection region can comprise one or more layers of a non-woven mesh of non-electrically conducting polymeric fibres that carry a permanent electrostatic charge otherwise known as an electret material. The electret material can be a polymer carrying a charge that is relatively stable to thermal decay for at least some months and preferably years. In one embodiment, the fibres can be polypropylene. One example of a suitable material is called ETR115 and is manufactured by Japan Vilene Company, Tokyo, Japan. Other suitable materials can be envisaged which do not have a static charge but are of a suitable nature to collect components of an airstream. As an example only, the suitable material can comprise a porous matt of fibrous material, such as cotton, or a sponge or sponge-like material.

In a further embodiment, the collection region can in addition be adapted to enable the collection of gaseous or other molecules of interest, including nitric oxide. In this embodiment, the collection region can additionally comprise a layer of activated carbon, alumina or silica. An as example only, the collection region can additionally comprise a layer of Trinitex® activated carbon filter media developed by Albstrom, Finland.

The collection region of the device may contain one layer or may contain a number of layers arranged in series, which allow both inhaled and exhaled air to pass through the same region. In one arrangement, the collection region can have a layer or layers adapted to preferentially collect a constituent of interest that is inhaled and another layer or layers can be adapted to preferentially collect a constituent of interest that is exhaled.

In another embodiment, the device may include one or more one-way valves mounted on the support member. Such one-way valve(s) may be adapted to at least partially or fully close on exhalation but at least partially of fully open on inhalation so forcing at least some or all of the exhaled airstream through the collection region resulting in at least some or all of the exhaled constituents being preferably captured by the collection region. By opening on inhalation, the subject can more readily inhale than would be the case if they had to inhale solely through the collection region. In another embodiment, the one-way valve(s) may be adapted to at least partially close on inhalation and at least partially open on exhalation so forcing at least some of the inhaled airstream through the collection region. In this arrangement, the subject can more readily exhale than would be the case if they had to exhale solely through the collection region.

In a further embodiment, the collection region can comprise a series of collection layers having different collection characteristics such that one type of constituent is collected in one layer and another type is collected in a different layer. In this embodiment, the series of collection layers can comprise the same material but have different collection characteristics. In another embodiment, two or more of the series of collection layers can comprise the same material. In yet a further embodiment, the series of collection layers can be formed respectively of different materials.

In one embodiment, the series of collection layers can have different porosities and so be adapted to collect particles of different sizes. For example, a first layer can be adapted to collect relatively larger sized droplets and/or particles but allow the passage of relatively smaller sized droplets and/or particles which are subsequently collected by another region that collects relatively small particles. This process can be repeated by a having a series of such layers with each layer being adapted to collect still smaller particles than the previous layer.

In another embodiment, a layer in the collection region can itself be made of different regions, with one region adapted to collect droplet particles and a different region adapted to collect relatively smaller molecules. As an example only, such an arrangement can be used to collect virus in mucous droplets in one region and collect molecules of clinical or pathological interest, such as exhaled NO gas, in another region.

In one embodiment, the at least one collection region can be removably mounted to the support member. In another embodiment, the at least one collection region can be non-removably mounted to the support member with the whole device constructed to enable any sample collected in the collection region to be analysed. In another embodiment, after use, the collection region, such as a layer of electret material or a matt or sponge, can be removed from the mask.

Once removed, the at least one collection region can undergo analysis to determine the nature of the constituents that have been collected. For example, the collection region can be placed in a tube with a buffer suitable for both penetration of the material making up the collection region (e.g. electret is often hydrophobic) and extraction of viral RNA for analysis by RT-PCR or separate solutions may be used in series.

In a further embodiment, the device can be entirely disposable. In another embodiment, the support member can be reusable with the collection region being mounted to the support member prior to use and then removed following use to allow analysis of the collected samples. It is anticipated that if some or all of the device is re-used, it would be adequately cleaned between respective uses so as to prevent cross-contamination.

In one embodiment, the collection region can comprise less than 50% of the width of the support member, more preferably less than 40% of the width of the support member. Still further, the collection region can comprise less than 50% of the height of the support member, more preferably less than 40% of the height of the support member. In another embodiment, the dimensions of the collection region can be optimized based on performance; determined by airflow through the region and the requirement of the analytical system. Where the collection region is disposed within or at end of a tube, the collection region may span all of the diameter of the tube or a portion of it.

The device can further comprise an attachment mechanism to facilitate mounting of the device over the nose and/or mouth of the subject. In one embodiment, the attachment mechanism can comprise two straps that extend respectively from spaced locations on the support member and which can pass around the back of the head and be tied together or be looped around the ears of the subject and hold the device in contact with the face. In another embodiment, one or more elastic members can extend from the support member and serve to hold the device in place in use. In another arrangement, the support member can be adapted to be held to the face by the subject when they are about to cough. This so-called “cough collector” device need not require an attachment mechanism.

The support member of the device can be constructed from many different materials and may have different shapes and appearances. It is preferred that the device fit relatively closely to the face so that the majority of inhaled or exhaled air (as desired) passes through the collection region. The support member can be of a semi-rigid construction or it may be of a soft and flexible construction. The materials used for the construction of the support member can have a relatively low or no permeability to air flow so that the exhaled or inhaled air is required to pass through the collection region, or the collection region and the valves if the latter are incorporated in the device.

In one embodiment, the location of the collection region on the support member is such that the collection region is exposed to the exhaled aerosols, particles and/or molecules but is not able to be contaminated by contact with non-aerosol biological material. An example of such contamination would be virus on the surface of the skin of the subject. Another example would be liquid mucous flowing from the nose that over the period of use which comes in contact with the collection surface. It is preferred that a gap exists between the skin of the subject and the location of the collection region.

Where the support member is rigid or semi-rigid, a gap between the skin of the subject and the collection region can be maintained by the shape of the support member. Even where the support member is rigid or semi-rigid or where the support member is formed from a relatively soft and/or flexible material, at least one layer of protective material can extend over at least the collection region. The layer of protective material can extend over the inner surface of the collection region, the outer surface of the collection region or both the inner and outer surface. Where present, the layer of protective material preferably prevents contamination of the collection region through physical contact by either the subject or others handling the device. The layer of protective material can be porous, inert and adapted to not collect molecules, particles or organisms in the airstream so they can be collected by the collection region. An example of a layer of protective material includes a layer of non-woven polypropylene although many other porous materials would be suitable. The layer of protective material may also be covered with a second protective film that is both highly porous and relatively highly hydrophobic in nature, with the purpose of this film being to prevent penetration of condensation and other liquid into the collection region.

Where the support member is semi-rigid, the support member can be moulded in one shape and then be deformable to fit closely to the face with no or only relatively small gaps. In another embodiment, the support member can be formed from a foldable material. In this embodiment, the support member can be flat-packed and then folded into an arrangement suitable for mounting over the mouth and/or nose of the subject. Where necessary, the support member can have portions that are self-adhesive or have adhesive portions provided thereon to allow construction of the support member into the necessary arrangement so that it is suitable for use.

The volume within the support member is preferably less than about 100 ml when the support member is mounted to the face of the subject. If the volume is much greater than this, it is anticipated that this space within the support member will provide an opportunity for exhaled air to mix within the mask and, what will be exhaled will be diluted with previously inhaled air from the dead space. It will also allow for condensation on the inner surface. These may reduce the sampling efficiency of the collection device.

In another embodiment, the support member can be made of an impermeable and flexible or stretchable material. Examples of suitable material include polymeric materials, such as vinyl polymers, such as polyvinyl chloride and closed-cell foams. In this embodiment, the cut or construction of the mask cloth can be such to allow the support member to appropriately adapt shape to fit the face of the subject. Instead or in addition, at least some or all of the edges of the cloth may be provided with a layer of adhesive, that can be protected with a removable barrier before placement, such that the edges of the mask can at least temporarily seal with the contact area of the face.

In a still further embodiment, the collection region can be covered with a removable barrier layer while within its delivery packaging and prior to use. This would typically be removed just prior to use.

In yet another embodiment, the collection region can be enveloped within the support member after use. This can be achieved, for example, by folding the support member such that the collection region is enclosed within the support member. This preferably serves to prevent contamination of the collection region until such time as the collection region undergoes analysis.

In a still further embodiment, collection device can be suitable for transportation after use to a location remote from its use so that the collection region can undergo analysis in an appropriate location, such as a diagnostic laboratory. For example, the collection device can be suitably packaged and transported through the postal service.

In a further aspect, the present invention is, a method of collecting constituents present in an airstream exhaled by a subject, comprising:

positioning a support member of a collection device over the nose and/or mouth of the subject, said support member mounting at least one collection region; and

collecting constituents of the exhaled airstream in the at least one collection region; and

holding said constituents in the collection region in a manner that allows subsequent analysis of said constituents;

wherein when the support member is mounted over the nose and/or mouth of the subject, at least some of said exhaled airstream passes through said at least one collection region.

In this aspect, the collection device can have one or more of the features of the collection device described herein.

In either aspect, the collection device (such as a mask, mask-like device or oro-nasal device) can be worn by the subject for a period of time that is determined by many factors including the threshold sensitivity of the analysis to be applied, the nature of the events that are being monitored, the risks of exposure that the subject may be subjected to as a consequence of the exposure and other practical factors that may arise, such as the health, comfort or tolerance of the subject. In one embodiment, the device can be worn between 5 minutes and 2 hours, more preferably between 15 mins and 1 hour and still more preferably for about 30 minutes. The subject may be asked to talk or read aloud for at least some or all of the time that the device is being worn by the subject. In another embodiment, the subject may be asked to monitor the number of times that they cough or leave the device in place until a predetermined number of coughs have occurred. The device may also be only positioned over the mouth and/or nose just prior to coughing and removed thereafter.

In both aspects, a range of analyses can be applied to the collection region following use to determine the nature of the collected constituents within the exhaled airstream. The analysis method can include PCR assay of DNA, RT-PCR for RNA followed by PCR, culture, ELISA immunoassays for various antigens (allergens, proteins, and inflammatory cytokines), or HPLC for gases. Many other forms of chemical and biochemical analysis from the fields of chemistry, proteomics, genomics and lipidomics and microbiology could be applied. The latter includes various bioassays by bacterial, cellular or fungal culture and plaque assays for viruses.

Multiple and different molecules, particles and/or microorganisms can be collected and analysed from one sample depending on the methods used. The analysis method can involve either or both culture techniques and RT-PCR/PCR methods and/or other methods drawn from the technologies of proteomics, genomics and analytical chemistry, biochemistry and immuno-pathology. One particular example is to concurrently measure normal commensurate organisms and other organisms of potential pathogenic interest. In this way, the commensurate organisms serve as a positive control showing that a sample from the region of the airway the commensurate organism colonises has been collected.

While suitable for humans, it will be appreciated that the collection device could be used on any animal subject where there are inhaled or exhaled particles or molecules of interest.

The present invention can be used to assist in understanding what microbial and non-microbial aerosols people generate, particularly when they may have a disease, such as a viral infection. For example, it is anticipated that the present invention can assist in understanding when a person is shedding a pathogenic organism, how much of the organism is shed at different times during the course of different respiratory infections, what is the particle size of the droplets carrying the organism when it is exhaled, and what are the amounts of the organism that are shed during different respiratory manoeuvres such as coughing, talking, sneezing or breathing.

Additionally, in many chronic respiratory diseases such as chronic obstructive pulmonary disease (COPD), cystic fibrosis (CF), allergic broncho-pulmonary aspergillosis (ABPA) and chronic asthma the persons may have persistent respiratory infections, multiple infections or infections that are resistant to the drugs used. In these cases it is anticipated that the present invention will assist in recovering samples of mucous in a far less invasive manner and so improve understanding about the nature of the infections present in one or more parts of the airways.

### BRIEF DESCRIPTION OF THE DRAWINGS

Hereinafter, by way of example only, embodiments of the invention are described with reference to the accompanying drawings, in which;

FIGS. 1 and 2 are views of one embodiment of a collection device according to the present invention;

FIGS. 3, 4 and 5 are views of another embodiment of a collection device according to the present invention;

FIG. 6 is a perspective view of a still further embodiment of a collection device according to the present invention;

FIG. 7 is a view of yet another embodiment of a collection device according to the present invention; and

FIG. 8 is a sectional view of one example of a collection region of a collection device according to the present invention.

### PREFERRED MODE OF CARRYING OUT THE INVENTION

Embodiments of the collection device and examples of use of such devices are depicted in the drawings.

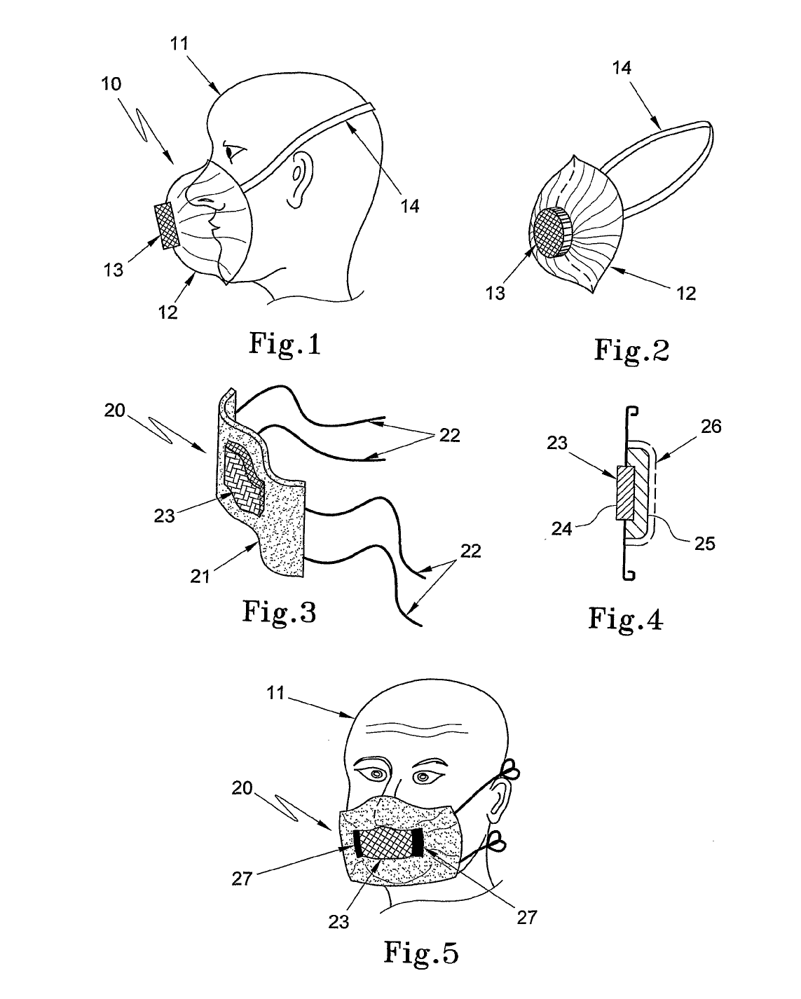

One embodiment of the collection device according to the present invention is depicted generally as 10 in FIGS. 1 and 2. In this embodiment, the collection device 10 is a semi-rigid mask for collecting constituents present in an airstream exhaled by a subject 11. The collection device comprises a support member 12 mountable over the nose and/or mouth of the subject and at least one collection region 13 mounted to the support member 12 for collecting at least some of the constituents in a manner that allows subsequent analysis of the constituents.

The device 10 is constructed such that when the support member is mounted over the nose and/or mouth of the subject 11, at least some and preferably all of the exhaled airstream is required to pass through the collection region 13 if it is to exit the device.

The device 10 is adapted to collect molecules, particles and/or organisms, such as respiratory droplets and aerosols, present in the exhaled airflow of the subject 11.

In the depicted embodiment, the collection region 13 comprises a number of layers of an electret material, namely a polymeric material coded as ETR115 and which is manufactured by Japan Vilene Company, Tokyo, Japan. Other suitable electret materials can be envisaged which do not have a static charge but are of a suitable nature to collect components of an airstream. As an example only, the suitable material can comprise a porous matt of fibrous material, such as cotton, or a sponge or sponge-like material.

If desired, the collection region 13 can in addition be adapted to enable the collection of gaseous or other molecules of interest, including nitric oxide. In this arrangement, the collection region 13 can additionally comprise a layer of activated carbon, alumina or silica. An as example only, the collection region can additionally comprise a layer of Trinitex® activated carbon filter media developed by Alhstrom, Finland.

Where the collection region 13 comprises a plurality of layers, one layer or a number of layer can be adapted to preferentially collect a constituent of interest, including a constituent that is inhaled or exhaled, and another layer or layers can be adapted to preferentially collect a constituent, such as a different constituent of interest, that is exhaled.

In the embodiment depicted in FIGS. 1 and 2, the support member 12 is formed from an air-impermeable material, for example a polyvinyl chloride and incorporates a head strap 14.

FIGS. 3-5 depict another arrangement of the collection device according to the present invention as 20. Here the support member 21 is made of relatively soft and flexible material and has a series of tie strings 22 to ensure appropriate positioning of the device 20 on the subject 11. In this embodiment, the collection region 23 is comprised of a number of portions.

The collection region 23 firstly comprises a layer of electret material as described herein 24 which is enclosed on at least its inner surface with a protective and non-collecting porous layer 25. The function of the layer 25 is to protect the electret material 24 from direct contamination by non-airborne sample, such as liquid mucous, or from direct contact with the skin of the subject 11. As depicted, a further protective layer 26 can be provided on the inner surface. The layer 26 can be both relatively highly porous and relatively highly hydrophobic in nature with its purpose being to prevent or at least partially prevent penetration of condensation and other liquid into the electret layer. If desired, the layer 26 could extend around the outside of the electret 24. It will be appreciated that a protective layer could also be provided on the outer surface.

In FIG. 5, the device 20 is depicted being worn by the subject 11. It will be noted from this figure that the mask fits relatively tightly to the face of the subject 11 such that the exhaled air passes through the collection region 23. In FIG. 5, the device 20 is slightly modified from that depicted in FIG. 3 in that it is also shown having two valves 27, one on either side of the electret 24. The depicted valves 27 are adapted to close on exhalation so forcing the airflow through the electret 24 and at least partially open on inhalation so allowing the subject 11 to more readily inhale than would otherwise be the case.

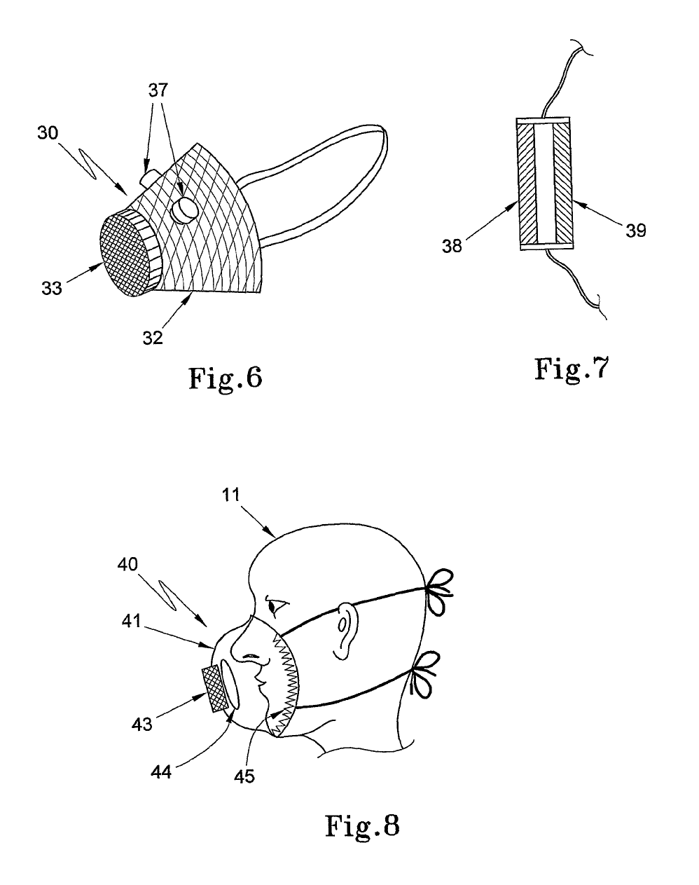

FIG. 6 depicts a still different arrangement of a device as 30. Here the device 30 is of semi-rigid construction and again has an electret collection region 33. The support member 32 of the device 30 is fitted with two one-way valves 37. The depicted valves 37 allow air into the mask on inhalation, but close on exhalation so forcing the exhaled air to be sampled by the collection region 33.

FIG. 8 depicts another embodiment of a collection device as 40. The device 40 has a support member 41 that has been cut from flexible impermeable cloth or foam, such as a closed-cell foam, and then folded to provide a cup-shaped support member.

The device 40 again has an electret collection region 43 and a porous layer 44 that separates the mouth and nose of the subject 11 from the collection region 43. A strip of adhesive film 44 is provided around the edges of the support member 41 to improve the seal to the face of the subject 11.

In this embodiment, the device 40 can be flat-packed and then folded into an arrangement suitable for mounting over the mouth and/or nose of the subject. Where necessary, the support member can have portions that are self-adhesive or have adhesive portions provided thereon to allow construction of the support member into the necessary arrangement so that it is suitable for use.

FIG. 7 depicts a cross section of one type of multilayered collection region mounted to a support member (only partially shown) for use in the collection regions depicted in the various embodiments, for example collection regions 13, 23, 33 and 43. The multilayered collection region is depicted with two layers 38 and 39. It will be appreciated that the layers 38 and 39 can be made of different materials and have different purposes. For example, the layer that is placed closest to the face of the subject 11 could comprise a layer of electret material to collect droplet aerosols and the other layer could be an activated carbon filter to collect a respired gas such as nitric oxide.

In another embodiment, the respective layers 38,39 can have different porosities and so be adapted to collect particles of different sizes. For example, one of the layers can be adapted to collect relatively larger sized droplets and/or particles but allow the passage of relatively smaller sized droplets and/or particles which are subsequently collected by another region that collects relatively small particles. This process can be repeated by a having a series of such layers with each layer being adapted to collect still smaller particles than the previous layer.

In another embodiment, one of the layers in the collection regions 13, 23, 33, 43 can itself be made of different regions, with one region adapted to collect droplet particles and a different region adapted to collect relatively smaller molecules. As an example only, such an arrangement can be used to collect virus in mucous droplets in one region and collect exhaled NO gas in another region.

In the various depicted embodiments, the collection regions can be removably or non-removably mounted to the various support members. As an example only, after use, the depicted collection regions can be cut or otherwise removed from the devices. Where the collection region is an electret, once removed, the collection region can undergo appropriate analysis to determine the nature of the collected constituents. As an example only, the collection region can be placed in a tube with a buffer suitable for both penetration of the electret (which is often hydrophobic) and extraction of viral RNA for analysis by RT-PCR.

The depicted collection devices can be entirely disposable or can be at least partially reusable with the collection region being mounted to the support member prior to use and then removed following use to allow analysis of the collected samples. It is anticipated that if some or all of the device is re-used, it would be adequately cleaned between respective uses so as to prevent cross-contamination.

The collection region will typically not comprise the entire device but instead can comprise less than 50% of the width of the support member, more preferably less than 40% of the width of the support member. Still further, the collection region can comprise less than 50% of the height of the support member, more preferably less than 40% of the height of the support member.

While the depicted embodiments have straps, in another arrangement, the support member can be adapted to be held to the face by the subject when they are about to cough. This so-called “cough collector” device need not require straps, or if present need not be used.

The volume within the support member of the depicted devices can be less than about 100 ml when it is fitted to the face of a subject. Greater than this volume and it is anticipated that this space within the support member will provide an opportunity for exhaled air to mix within the mask and what will be exhaled will be diluted with previously inhaled air from the dead space. It will also allow for condensation on the inner surface. These may reduce the sampling efficiency of the collection device.

In the arrangement depicted in FIG. 8, the collection region 43 can be enveloped within the foldable support member 41 after use. This preferably serves to prevent contamination of the collection region 43 until such time as the collection region 43 and the constituents captured therein undergo analysis. In this regard, once folded, the collection device can be in a form that is suitable for transportation after use to a location remote from its use so that the collection region 43 can undergo analysis in an appropriate location, such as a diagnostic laboratory. For example, the collection device 40 can be suitably packaged and transported through the postal service.

As an alternative to the depicted arrangements, the device may include a member, such as a tube or tubes, that are fitted into an oral and/or nasal cavity. Where used, the tube can feed an exhaled airstream to the at least one collection region. In this embodiment, the at least one collection region can be positioned within the tube, at a proximal or distal end of the tube or be positioned external the tube on the support member.

In use, the support member of the various collection devices depicted herein are positionable over the nose and/or mouth of the subject so as to allow collection of selected constituents in the exhaled airstream of the subject. The collection region works to hold the constituents in a manner that allows subsequent analysis of these constituents.

In use, the collection device will be worn by the subject for a period of time that is determined by many factors including the threshold sensitivity of the analysis to be applied, the nature of the events that are being monitored, the risks of exposure that the subject may be subjected to as a consequence of the exposure and other practical factors that may arise, such as the health, comfort or tolerance of the subject. In one embodiment, the device can be worn between 5 minutes and 2 hours, more preferably between 15 mins and 1 hour and still more preferably for about 30 minutes. The subject may be asked to talk or read aloud for at least some or all of the time that the device is being worn by the subject. In another embodiment, the subject may be asked to monitor the number of times that they cough or leave the device in place until a predetermined number of coughs have occurred. The device may also be only positioned over the mouth and/or nose just prior to coughing and removed thereafter.

A range of analyses can be applied to the collected samples. The analysis method can include PCR assay of DNA, RT-PCR for RNA followed by PCR, culture, ELISA immunoassays for various antigens (allergens, proteins, and inflammatory cytokines), or HPLC for gases. Many other forms of chemical and biochemical analysis from the fields of chemistry, proteomics, genomics and lipidomics and microbiology could be applied. The latter includes various bioassays by bacterial, cellular or fungal culture and plaque assays for viruses.

Multiple and different molecules, particles and/or microorganisms can be collected and analysed from one sample depending on the methods used. The analysis method can involve either or both culture techniques and RT-PCR/PCR methods and/or other methods drawn from the technologies of proteomics, genomics and analytical chemistry, biochemistry and immuno-pathology. One particular example is to concurrently measure normal commensurate organisms and other organisms of potential pathogenic interest. In this way, the commensurate organisms serve as a positive control showing that a sample from the region of the airway the commensurate organism colonises has been collected.

While suitable for humans, it will be appreciated that the collection device as depicted herein could be used on any animal subject where there are inhaled or exhaled particles or molecules of interest.

The present invention can be used to assist in understanding what microbial and non-microbial aerosols people (or animals) generate, particularly when they may have a disease, such as a viral infection. For example, it is anticipated that the present invention can assist in understanding when a person (or animal) is shedding a pathogenic organism, how much of the organism is shed at different times during the course of different respiratory infections, what is the particle size of the droplets carrying the organism when it is exhaled, and what are the amounts of the organism that are shed during different respiratory manoeuvres such as coughing, talking, sneezing or breathing.

Additionally, in many chronic respiratory diseases such as chronic obstructive pulmonary disease (COPD), cystic fibrosis (CF), allergic broncho-pulmonary aspergillosis (ABPA) and chronic asthma the persons may have persistent respiratory infections, multiple infections or infections that are resistant to the drugs used. In these cases it is anticipated that the present invention will assist in recovering samples of mucous in a far less invasive manner and so improve understanding about the nature of the infections present in one or more parts of the airways.

### Example 1

One example of use of a collection device according to the present invention is provided below.

### Methods and Materials

Twenty young adult volunteers (4 male and 6 female), with a mean duration of symptoms of 33 days, were selected using a modified common cold questionnaire. They each provided a sample of nasal mucus and then wore a separate sampling mask on each of the following 3 occasions: 20 min of reading aloud; 20 min of quiet breathing, and 20 voluntary coughs over a 3-5 min period. These times were a compromise between the time that was anticipated to be sufficient and the time that would be tolerated by the patient. At no time during sampling did patients or researchers touch the collection surface of the mask with their gloved hands; only the ties were used to hold the mask to the face were touched. Volunteers who had no symptoms of a cold for at least the previous 2 weeks (n=3) were recruited as negative control subjects.

The sampling device consisted of a hand-made close fitting, half-face mask made from an impermeable, stretchable PVC that contained a central section of electret (diameter, 25 mm; ETR115; Japan Vilene) that was located opposite the nose and mouth region. Testing indicated it collected >80% of 0.52 μm latex particles (virus droplet proxy) and had low airflow resistance (<15 mm H₂O) at normal respiratory flows. After sampling, the electret was immediately removed from the mask and was placed in 1 mL of RNA lysis buffer (Qiagen); it was then transported at −80° C. to the laboratory, and RNA was isolated using the Qiagen RNeasy Mini Kit (Qiagen) according to the manufacturer's instructions. Following conversion to cDNA, rhinovirus and respiratory syncytial virus were assessed by an in-house PCR assay using 50 cycles of amplification. Subsamples from 9 patients who had more severe symptoms were also tested for the presence of influenza A and B virus, parainfluenza virus 1, 2, and 3, respiratory syncytial virus, and human metapneumovirus using a single-round, in-house multiplexed PCR assay, based on primers. Results were confirmed using specific biotin-labelled oligonucleotides in a multiplexed assay.

### Results

The detection of viruses in the different samples by PCR is shown in Table 1. Of the 20 patients who had samples tested, 9 had virus-positive nasal mucus samples. Rhinovirus was detected in samples from 6 of 20 patients, influenza A virus was detected in samples from 2 of 9 patients, and parainfluenza virus 3 was detected in samples from 2 of 9 patients. Parainfluenza viruses 1 and 2, respiratory syncytial virus, and human metapneumonvirus were not detected in any of the samples. Of the 9 patients who had virus-positive mucus samples, 6 had ≧1 virus-positive aerosol sample. Six patients had virus-positive samples from coughing, 5 from talking, and 3 from breathing. The aerosol sample from patient 6 tested positive for both rhinovirus and parainfluenza, although the mucus sample from this patient was only positive for rhinovirus. None of the asymptomatic subjects had a sample that tested positive for virus. There were no major differences in detection of viruses between male and female subjects.

### Discussion

This study led to direct detection of virus aerosols exhaled by infected individuals. The study reveals that breathing alone, for some people, is sufficient to aerosolize rhinovirus.

Although coughing, talking and sneezing have conventionally been associated with the generation of virus aerosols, an association that was confirmed in this study-generation by breathing alone had not been previously indicated for humans. Whether this is confined to rhinovirus is now known, because only a few samples were examined in this study.

TABLE 1
Results of PCR of exhaled virus aerosols generated by coughing,
talking and breathing for 9 patients whose nasal mucus samples
tested positive for at least 1 virus.

| | Virus | Nasal | | | | Day of |
|---|---|---|---|---|---|---|
| Patient | Detected | mucus | Coughing | Talking | Breathing | illness |
| 1 | Rv | + | + | − | − | 2 |
| 2 | Rv | + | + | + | + | 2 |
| 3 | Rv | + | + | + | + | 5 |
| 4 | Rv | + | − | + | − | 3 |
| 5 | Rv | + | + | − | − | 4 |
| 6 | Rv | + | + | + | + | 2 |
| 6 | Para | − | + | + | − | 2 |
| 7 | Para | + | + | + | − | 4 |
| 8 | Flu | + | − | − | − | 5 |
| 9 | Flu | + | − | − | − | 3 |
| Total | . . . | 9 | 7 | 6 | 3 | 3.3^{a} |

Note:
Flu, influenza virus;
−, negative;
Para, parainfluenza virus;
+, positive;
Rv, human rhinovirus.
^{a}Mean value.

The sampler tested in this example is intuitive to use, safe, simple, non-invasive, suitable for a wide range of age groups, easily stored, and inexpensive. Use of the electret is safer for clinical staff than are current procedures that require handling of secretions. The combination of a mask with multiplexed PCR provides a tool with a wide range of applications in the diagnosis and management of respiratory infections.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

1. A collection device for collecting constituents present in an airstream exhaled by a subject, the collection device comprising:
a support member mountable over the nose and/or mouth of the subject; and
at least one collection region mounted to the support member for collecting at least some of said constituents and holding said constituents in the collection region in a manner that allows subsequent analysis of said constituents;
wherein when the support member is mounted over the nose and/or mouth of the subject, at least some of said exhaled airstream passes through said at least one collection region.

2. The collection device of claim 1 wherein the collection device is a mask, a mask-like device, or an oro-nasal device.

3. The collection device of claim 2 wherein the constituents comprise respiratory droplets, aerosols and/or other particles being exhaled by the subject.

4. The collection device of claim 2 wherein the collection region comprises one or more layers of a non-woven mesh of non-electrically conducting polymeric fibres that carry a permanent electrostatic charge.

5. The collection device of claim 1 wherein the collection region comprises a porous mesh or matrix that collects constituents of interest from the exhaled airstream.

6. The collection device of claim 2 wherein the constituents comprise gaseous or other molecules of interest or viruses, fungi or bacteria present in the exhaled airstream.

7. The collection device of claim 6 wherein the collection region comprises a layer of activated carbon, alumina or silica.

8. The collection device of claim 2 wherein the collection region comprises a plurality of layers arranged in series and through which the exhaled airstream is required to pass.

9. The collection device of claim 8 wherein at least one layer preferentially collects a constituent that is inhaled by the subject and wherein at least another layer preferentially collects a constituent that is exhaled by the subject.

10. The collection device of claim 8 wherein each layer in the collection region is adapted to collect different constituents to the other layers.

11. The collection device of claim 10 wherein each layer in the collection region comprises the same material but formed to have a different collection characteristic to the other layers.

12. The collection device of claim 8 wherein each layer in the collection region has a different porosity to that of the other layers.

13. The collection device of claim 12 wherein the porosity of each layer in the collection region decreases compared to the previous layer through which an exhaled airstream is required to pass.

14. The collection device of claim 1 wherein at least one layer in the collection region is formed of different regions, with a first region being adapted to collect droplet or aerosol particles and at least a second region adapted to collect relatively smaller molecules.

15. The collection device of claim 14 wherein the first region collects virus in mucous droplets and said second region collects molecules of clinical or pathological interest.

16. The collection device of claim 1 further comprising one or more one-way valves mounted on the support member.

17. The collection device of claim 16 wherein the valve(s) at least partially or fully close on exhalation so forcing all of the exhaled airstream through the collection region, and then at least partially open on inhalation.

18. The collection device of claim 1 wherein the at least one collection region is removably mounted to the support member.

19. The collection device of claim 2 further comprising attachment means to facilitate mounting of the device over the nose and/or mouth of the subject.

20. The collection device of claim 1 wherein the support member is of a rigid or semi-rigid construction or of a soft and flexible construction and wherein the material(s) used for the construction of the support member have a relatively low or no permeability to air flow.

21. The collection device of claim 20 wherein where the support member is rigid or semi-rigid, the at least one collection region is mounted on the support member so that there is a gap between the skin of the subject and the collection region.

22. The collection device of claim 20 wherein at least one layer of protective material extends over at least the collection region.

23. The collection device of claim 22 wherein said at least one layer of protective material extends over an outer surface of the collection region.

24. The collection device of claim 22 or 23 wherein said at least one layer of protective material is porous, inert and/or adapted to not collect said constituents in the airstream so they can be collected by the collection region.

25. The collection device of claim 20 wherein the support member is moulded in one shape and is then deformable to fit closely to the face of the subject with no or only relatively small gaps.

26. The collection device of claim 20 wherein the volume within the support member is less than about 100 ml when the device is fitted to the face of the subject.

27. The collection device of claim 20 wherein the construction material of the support member is a vinyl polymer, such as polyvinyl chloride, or a closed cell foam.

28. The collection device of claim 27 wherein at least some or all of the edges of the support member are provided with a layer of adhesive such that the edges of the support device can at least temporarily seal with the face of the subject.

29. A method of collecting constituents present in an airstream exhaled by a subject, comprising:
positioning a support member of a collection device over the nose and/or mouth of the subject, said support member mounting at least one collection region; and
collecting constituents of the exhaled airstream in the at least one collection region; and
holding said constituents in the collection region in a manner that allows subsequent analysis of said constituents;
wherein when the support member is mounted over the nose and/or mouth of the subject, at least some of said exhaled airstream passes through said at least one collection region.

30. The method of claim 29 wherein the collection device is worn by the subject for a between 5 minutes and 2 hours, more preferably between 15 mins and 1 hour, and still more preferably for about 30 minutes.

31. The method of claim 29 wherein said subsequent analysis is selected from the group comprising PCR assay of DNA, RT-PCR for RNA followed by PCR, culture, ELISA immunoassays for various antigens (allergens, proteins, and inflammatory cytokines), HPLC for gases, and chemical and biochemical analyses from the fields of chemistry, proteomics, genomics and lipidomics and microbiology including various bioassays by bacterial, cellular or fungal culture and plaque assays for viruses.
